SEQUENCE LISTING (7 sequences)

SEQ ID NO: 1 - DNA, 13 bp, synthetic construct
  misc_feature 1..13: note = Synthetic construct
  source 1..13: mol_type = other DNA
ggctaaactgggc

SEQ ID NO: 2 - DNA, 11 bp, synthetic construct
  misc_feature 1..11: note = Synthetic Construct
  source 1..11: mol_type = other DNA
gctatactggc

SEQ ID NO: 3 - DNA, 11 bp, synthetic construct
  misc_feature 1..11: note = Synthetic Construct
  source 1..11: mol_type = other DNA
ggctaaacggc

SEQ ID NO: 4 - DNA, 12 bp, synthetic construct
  misc_feature 1..12: note = Synthetic Construct
  source 1..12: mol_type = other DNA
ggctaactgggc

SEQ ID NO: 5 - DNA, 12 bp, synthetic construct
  misc_feature 1..12: note = Synthetic Construct
  source 1..12: mol_type = other DNA
gctaaactgggc

SEQ ID NO: 6 - DNA, 13 bp, synthetic construct
  misc_feature 1..13: note = Synthetic Construct
  source 1..13: mol_type = other DNA
ggctaaactgggc

SEQ ID NO: 7 - DNA, 12 bp, synthetic construct
  misc_feature 1..12: note = Synthetic Construct
  source 1..12: mol_type = other DNA
gccaaactgggc

(19) United States
(12) Patent Application Publication
(10) Pub. No.: US 2023/0298701 A1
(43) Pub. Date: Sep. 21, 2023

(54) DEEP-LEARNING-BASED TECHNIQUES FOR GENERATING A CONSENSUS SEQUENCE FROM MULTIPLE NOISY SEQUENCES

(71) Applicant: Roche Sequencing Solutions, Inc., Pleasanton, CA (US)

(72) Inventors: Marghoob MOHIYUDDIN, Pleasanton, CA (US); Sayed Mohammadebrahim SAHRAEIAN, Pleasanton, CA (US)

(21) Appl. No.: 18/113,308

(22) Filed: Feb. 23, 2023

Related U.S. Application Data
(63) Continuation of application No. PCT/US2021/049561, filed on Sep. 9, 2021.
(60) Provisional application No. 63/077,357, filed on Sep. 11, 2020.

(51) Int. Cl. G16B 30/10; G16B 40/20; G06N 3/0442; G06N 3/08

(52) U.S. Cl. CPC G16B 30/10; G06N 3/0442; G06N 3/08; G16B 40/20

(57) ABSTRACT

Some embodiments relate to methods, systems, uses, or software for generating a consensus sequence of a particular molecule. A set of sequences of the particular molecule can be accessed, each having been generated independently from other sequences in the set of sequences and each including an ordered set of bases. An alignment process may be performed using the set of sequences to generate an alignment result associating, for each base of the ordered sets of bases of the sets of sequences. The base may have a reference position. For each reference position of a set of reference positions, a feature vector for the reference position may be generated that represents each base from the ordered sets of bases aligned to the reference position. The feature vectors for the set of references positions may be processed using a machine learning model to generate the consensus sequence for the particular molecule.

### CROSS-REFERENCE TO RELATED APPLICATIONS

This application is a bypass continuation of International Application No. PCT/US2021/049561 filed Sep. 9, 2021, which claims the benefit of and priority to U.S. Provisional Pat. Application 63/077,357, filed on Sep. 11, 2020, both of which are hereby incorporated by reference in their entireties for all purposes.

### SEQUENCE LISTING

The instant application contains a Sequence Listing which has been submitted electronically in txt file format and is hereby incorporated by reference in its entirety. Said txt copy, created on Jan. 11, 2023, is named 1256945_ST25.txt and is 1,761 bytes in size.

### BACKGROUND

Biological samples can be used to identify a sequence that identifies ordered sets of nucleic acids. Rather recently, single-molecule sequencing has been rapidly advancing in techniques and expanding in applications. These techniques can sequence individual molecules and can be performed in real-time without PCR amplification. The techniques hold incredible promise with regard to facilitating: building of comprehensive libraries relating genes to diseases, identifying and characterizing new diseases, characterizing rare diseases, and identifying therapies.

However, many existing sequencing techniques (e.g., third-generation or next-generation techniques) remains susceptible to errors, with error rates that may reach 40%. The utility of sequencing would be highly increased if the error rate could be reduced.

### SUMMARY

In some embodiments, a computer-implemented method is provided for generating a consensus sequence of a particular molecule. A set of sequences of the particular molecule is accessed, each of the set of sequences having been generated independently from other sequences in the set of sequences, and each of the set of sequences including an ordered set of bases. An alignment process is performed using the set of sequences to generate an alignment result that associates, for each base of the ordered sets of bases of the sets of sequences, the base with a reference position from among a set of reference positions. For each reference position of the set of reference positions, a feature vector is generated for the reference position that represents each base from the ordered sets of bases aligned to the reference position. The feature vectors for the set of references positions are processed using a machine learning model to generate the consensus sequence for the particular molecule.

Performing the alignment processing may include performing multiple sequence alignment. For each reference position of the set of reference positions, the feature vector may include, for each of the set of sequences, an indication as to which, if any, of the ordered set of bases is aligned to the reference position. For each reference position of at least one reference position of the set of reference positions, the feature vector may include an indication that each of at least one of the set of sequences does not include a base aligned to the reference position. The method may further include, for each sequence of at least one of the set of sequences: determining that the sequence includes one or more homopolymers, each of the one or more homopolymers including multiple sequential representations of a same base in the sequence; and generating a collapsed representation of the sequence in which each of the one or more homopolymers is collapsed to a single base, wherein the alignment process is performed using the collapsed representations of the sequence. The collapsed representation may include, for each of the one or more homopolymers, an indication of a quantity of bases in the homopolymer. The machine learning model may include a recurrent neural network. The machine learning model may include one or more long short-term memory (LSTM) units. The method may further include accessing, for each sequence of at least some of the set of sequences, a quality metric for each of one or more bases of the ordered set of bases, where at least one of the generated feature vectors includes one or more quality values, each of the one or more quality values including or being based on the quality metric.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 depicts an exemplary sequence assessment network for generating consensus sequences.

FIGS. 2A-2D illustrate exemplary processing of sequences to generate a consensus sequence.

FIG. 3 illustrates an exemplary neural network for processing a representation of a set of sequences to generate a result corresponding to a consensus sequence.

FIGS. 4A-4D illustrate exemplary processing of sequences to generate a consensus sequence.

FIGS. 5A-5D illustrate exemplary processing of sequences to generate a consensus sequence.

FIG. 6 illustrates an exemplary neural network for processing a representation of a set of sequences to generate a result corresponding to a consensus sequence.

FIG. 7 illustrates a flowchart of an exemplary process for processing a set of sequences to generate a consensus sequence.

FIG. 8 shows exemplary consensus base-level Phred scores across cluster sizes for each of two techniques for generating consensus sequences.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Techniques disclosed herein can process multiple sequences pertaining to a same molecule, gene and/or sample to generate a consensus sequence. Each individual sequence may be prone to errors introduced during (for example) amplification, sample preparation, and/or sequencing. However, collective assessment of multiple sequences may reduce the probability that a resulting sequence includes any error and/or reduce (e.g., on average) a quantity of errors in the sequence.

The individual sequences may be independent from each other and/or may have been generated (for example) using different techniques, using different machines, using different sample portions, and/or at different times relative to each other. In other embodiments, the individual sequences may have been generated using the same sample, on the same machine, and at approximately the same time, such as through parallel sequencing through a next generation or third generation sequencer (i.e., a nanopore sequencer), for example. Each of the individual sequences may include an ordered set of bases (e.g., nucleic-acid bases). Each of the individual sequences may be of a particular molecule and may have a length that is less than (for example) 20,000 nucleotides, less than 15,000 nucleotides, less than 10,000 nucleotides, less than 5,000 nucleotides, less than 1,000 nucleotides, less than 500 nucleotides or less than 200 nucleotides.

In some instances, sequences may be longer and may have a length that is more than (for example) 20,000 nucleotides, more than 15,000 nucleotides, more than 10,000 nucleotides, more than 5,000 nucleotides, more than 1,000 nucleotides, more than 500 nucleotides or more than 200 nucleotides. In these situations, each of the sequences may be divided into multiple portions (e.g., each having a length that is less than or equal to 1,000 nucleotides, less than or equal to 500 nucleotides, less than or equal to 200 nucleotides or less than or equal to 100 nucleotides) or a sequence may be processed as a whole without partitioning. Corresponding portions of the set of sequences may then be collectively assessed in portion-specific manners to predict a consensus sequence for the portion. The consensus sequences for the portions may then be concatenated to form a consensus sequence for the sample.

The individual sequences may be aligned to each other using an alignment technique. The alignment technique may include multiple sequence alignment, which may use (for example) a progressive, iterative, Hidden-Markov and/or consensus technique to align the sequences. The alignment may include introducing gaps between at least some sequential nucleotides to account for insertion and/or deletion type errors.

In some instances, a modified representation of each sequence is generated and the modified representations are then aligned using an alignment technique. The modified representation of a sequence can be generated by detecting any homopolymer in the sequence (that includes multiple successive instances of a same base) and collapsing each homopolymer to a single base. For example, a sequence may include 3 consecutive guanines, which may be replaced by a single guanine in the modified representation.

Whether sequences are aligned or modified representations of the sequences are aligned, a result of the alignment can include - for each of a set of positions (which may represent individual bases and potentially homopolymers) - an indication as to which base each aligned sequence includes in association with the position.

A set of feature vectors can be generated based on the alignment result. For example, for each position in the alignment result, a feature vector may indicate - for each of the aligned sequences - which base (or gap) is aligned to the position. When condensed modified representations of the sequences are aligned and when a representation of a homopolymer is aligned to a given position, the feature vector may further identify a number of bases that were included in the homopolymer. In some instances, a feature vector further includes one or more quality metrics.

The feature vectors can then be processed by a machine learning model. The machine learning model may be configured to (for example) generate an output that represents a same number of positions as represented in the input and that identifies, for each position a particular nucleotide (or gap/null value) predicted to correspond to the positions. In some instances, the output further includes, for each position, a confidence metric associated with the position. A total size of the output of the model may be smaller than a size of the input, as the feature vectors each have a length greater than one, greater than two, etc.

The total model output may then be post-processed. For example, the output may be condensed to remove each gap/null value. A consensus sequence (e.g., for a particular molecule or particular sample) can then be defined to be or can be defined based on the post-processed output. The consensus sequence may be an intra-molecular consensus prediction (e.g., when a same molecule is repeatedly used to generate multiple different sequences such as a molecule generated through rolling circle amplification or a concatamer of repeat sequences) or an intermolecular consensus prediction (e.g., when different molecules of same or different types are used to generate multiple different sequences).

The consensus sequence may be transmitted (e.g., from a central server and/or central computing system) to another device, which may include a device associated with a care provider and/or laboratory. In some instances, the consensus sequence may be compared to each of one or more reference sequences to predict whether the consensus sequence includes any variants and/or mutations (e.g., single nucleotide variants), and a result based on the comparison may be transmitted to another device.

In some instances, the consensus sequence is used as a Unique Molecular Identifier (UMI), which may be attached to a given DNA fragment before PCR amplification. The UMI can then be used as a tag to identify each duplicate that corresponds to a particular fragment. Thus, reads originating from PCR duplicates may be identified by detecting the same UMIs, and reads associated with the UMIs can be collapsed, which can facilitate accurately estimate relative concentrations of fragments in the original sample.

In some instances, the consensus sequence can be used for sequence assembly refinement. For example, the set of reads that are clustered together to generate a contig during the assembly process, or the set of reads that are aligned to a given contig generated by the assembly process can be collapsed to generate the consensus contig and thus reduce the errors in the assembled contigs.

### II. Sequence Identification, Sequence Representations, Sequence Alignments, Feature Vectors and Machine Learning Model

### II.A. Sequence Identification

A sequence may be identified by processing a sample, which may include (for example) a blood, saliva, or tissue biopsy collected from a subject. Sequencing is emerging to include technologies for sequencing single molecules. Exemplary single-molecule sequencing techniques include nanopore sequencing, single-molecule real-time sequencing (SMRT), Illumina’s sequencing-by-synthesis and Helicos sequencing.

### II.A.1. Nanopore Sequencing

Nanopore sequencing includes imbedding a protein pore in a synthetic membrane. An ionic current can be passed through the nanopore. The nanopore can have a polymerase molecule near the pore entrance. Nucleotides with tags corresponding to the four nucleotide bases can be introduced. The polymerase molecule can incorporate the tagged nucleotides to create a sequence of base pairs. The nanopore can capture the corresponding tags as the DNA template strand is incorporated. The tags in the nanopore can cause a detectable change in the current or some other electrical property that can be measured. Each tag can cause a specific current change, so the nucleotide sequence can be identified.

Other methods of nanopore sequencing can involve threading the molecule to be sequenced directly through the pore. The molecule to be sequenced may be a nucleic acid molecule, a derivative molecule or modified molecule of a nucleic acid, or some other macromolecule that can be threaded through the pore.

Insertion and deletion errors and mismatches may be introduced during nanopore sequencing. Error rates may be reduced with more accurate base-calling algorithms, which can be used to identify the base sequence based on the current changes. Additionally, repeating the sequencing of a DNA molecule at a nanopore can create a consensus sequence and reduce the error rate of nanopore sequencing.

### II.A.2. Single-Molecule Real-Time Sequencing (SMRT)

Single-molecule real-time sequencing (SMRT) is a method of parallelized single-molecule DNA sequencing. Adapters can be added to the ends of a DNA or RNA molecule to convert a double stranded molecule to a single, circular template. A DNA polymerase enzyme can be attached to the template. During sequencing, the template DNA molecule can be placed in a zero-mode waveguide. As the polymerase incorporates the nucleotides, light can be emitted. The light emitted for each nucleotide base is different, so each nucleotide can be identified.

Errors can occur during SMRT sequencing as a result of insertions, deletions, and ‘dark bases’. Dark bases are bases that do not fluoresce during imaging, and therefore cannot be identified in the sequence. For a single-pass sequence, the error rate is around 11%. Taking multiple passes of a single template molecule and averaging the sequences can build confidence in a consensus sequence.

### II.A.3. Illumina Sequencing

Illumina sequencing is a sequencing-by-synthesis approach for DNA sequencing. Adapters can be added to the ends of DNA fragments and the DNA fragments can be hybridized on a flow cell. A DNA polymerase molecule can create a complement of each DNA fragment. The original DNA fragment can then be washed away. Bridge amplification can clonally amplify the complement strands to create clusters. After amplification, the reverse strands can be cleaved and washed off. During sequencing, fluorescent nucleotides can be introduced and compete for addition to the nucleotide chain. Thus, matching fluorescent nucleotides of a given type can be selectively added to form a base pair. A light source can be used to excite the fluorescent nucleotides so that a light signal is emitted. Different fluorescent nucleotides may be iteratively added (and subsequently imaged). Each nucleotide base can emit a different light signal, so that the nucleotide that was added can be determined.

Nucleotide substitutions cause the majority of the errors during Illumina sequencing. Other errors can result from cross-talk between the excitation and emission spectra, cross-talk between clusters, phasing, and dimming. A Phred quality score (Q) is defined for each base during Illumina sequencing. The quality score is defined using the equation Q = -10logio(P). Higher Q scores indicate a smaller probability (P) of an error, whereas lower Q scores indicates a higher likelihood of error. For example, a quality score of 20 represents an accuracy of 99%. Quality predictor values, which can be determined from intensity profiles and signal-to noise-ratios during the base determination, can be used to determine the quality score associated with a base. The Phred quality score and/or other types of quality scores can be applied to sequences generated from other sequencing approaches as well.

### II.A.4. Helicos Sequencing

To prepare for Helicos sequencing, each DNA molecule can be cut into lengths averaging around 35 base pairs. A primer can be added to a 3′ end of each DNA strand. Each strand, which will serve as a template for sequencing, can be labeled with a fluorescent nucleotide. The DNA strands can be hybridized to a flow cell. A laser can illuminate the flow cell, showing the location of each template. Images can be taken of each template, and then the fluorescent labels can be cleaved.

DNA polymerase and a fluorescent nucleotide corresponding to one of the four nucleotide bases can be added to the flow cell. The DNA polymerase can insert the fluorescent nucleotide for each template that has a nucleotide corresponding to the fluorescent nucleotide. For example, a DNA polymerase will add a T fluorescent nucleotide to each template with an A nucleotide next in the sequence. After imaging, the fluorescent label for the fluorescent nucleotide can be removed and a new fluorescent corresponding to another nucleotide can be introduced. The process can be repeated until the desired read length is achieved.

Helicos sequencing also suffers from errors, mainly as a result of dark bases and nucleotide substitutions. Similar to SMRT sequencing, repetitive sequencing can decrease the errors and increase the confidence in an identified base. However, repeated sequencing can increase the cost of the sequencing process.

### II.B. Sequence Representations

In some instances, a sequence representation may identify, for each of one or more positions, a base (e.g., nucleotide) present at that position in a particular sequence. For example, a sequencing output may identify a sequence having 75 nucleotides, such that a sequence representation identifies 75 nucleotides.

In some instances, each of one or more bases is further associated with a quality and/or confidence metric that may have been generated based on raw data underlying the base identification. For example, an optical signal may have been used to predict a corresponding base, and a frequency, intensity and/or pulse width may be used to identify a confidence of the predicted base (e.g., based on a predefined look-up table and/or relationship).

In some instances, an originally detected sequence includes one or more homopolymers that include multiple successive positions of a same base. A sequence representation may then include a collapsed representation that replaces each homopolymer with a single instance of the corresponding base. In some instances, the replacement value and/or a corresponding structure identifies a quantity of the base in each homopolymer.

In some instances, a sequence representation includes an encoded representation. For example, rather than a sequence representation including an “A” (or “C”, “G” or “T”), the encoding may include a binary or integer representation of a base. For example, each position may be associated with at least four or at least five binary values that are to be set to indicate whether a base at the position is an adenine, cytosine, guanine, thymine or potentially none of the above. Such encoding may correspond to (for example) a one-hot encoding. As another example, a sequence representation may be configured such that each of a set of integers (or a set of other characters) represents a particular nucleotide (e.g., and potentially a gap or null value).

### II.C. Sequence Alignments

To facilitate reducing sequence errors, a consensus sequence may be generated using a set of sequences. In some instances, at least some or all of the set of sequences may have been generated using a same sample, a same molecule, a same sequencing technique and/or a same sequencing device. Alternatively or additionally, at least some or all of the set of sequences may have been generated using different samples, different molecules, different sequencing techniques and/or a sequencing devices. As one illustrative example, the set of sequences may have been generated by using a same sequencing system and technique (e.g., nanopore sequencing) and using a same sample. A consensus sequence may be determined locally within the sequencing system and output to a user.

The set of sequence representations (which can include the sequences themselves or collapsed versions that replace each homopolymer with a single base) can be aligned to each other, which can facilitate generation of feature vectors. The alignment is performed using a cost or loss function. For example, each of a set of potential alignments can be evaluated be introducing a penalty for each instance where aligned bases are not the same and/or for each gap. An alignment associated with a lower total cost (i.e., sum of the penalties) may be preferentially selected over an alternative alignment associated with a higher total cost. In some instances, potential alignments are iteratively evaluated in a pair-wise manner.

In some instances, the sequences are aligned using a multiple sequence alignment technique. The multiple sequence alignment technique may use (for example) progressive alignment construction (e.g., that first uses a clustering technique to generate a guide tree that identifies relationships between sequences and that then iteratively combines alignments in a manner that begins with a most-similar pair and progresses to repeatedly align a next-most similar sequence). Exemplary techniques implementing a progressive method include one or more versions in the Clustal family (e.g., ClustalW) and/or T-Coffee. The multiple sequence alignment technique may include an iterative method, which may again use a guide tree and iteratively sequentially adding sequences to the alignment. However, unlike the progressive techniques, an iterative method can include repeatedly realigning the initial sequences. An iterative method may (for example) use a hill-climbing algorithm for the realignment (e.g., as implemented in PRRN/PRRP), need not include a gap penalty (e.g., as implemented in DIALIGN), and/or may use a distance metric to identify sequence similarity (e.g., as used in the MUSCLE technique). The multiple sequence alignment technique may use a Hidden Markov model, which can generate probabilities of particular proposed alignments (e.g., based on a likelihood of base differences, base consistency and/or gap occurrences), such that an alignment associated with a high or highest probability may be selected. Exemplary Hidden-Markov techniques are used in Sequence Alignment and Modeling System (SAM) software and HMMER software. The multiple sequence alignment technique may use a phylogeny-aware method, such as PRANK or ProGraphMSA.

### II.D. Feature Vectors

Aligned sequence representations may thus have multiple dimensions, including a first dimension corresponding to positions and a second dimension corresponding to individual sequences. A feature vector may be generated for each of the positions represented in the alignment. The feature vector associated with a given position may indicate the bases (and/or gaps) associated with the given position across the set of sequences.

Nucleotides may be indicated using (for example) one-hot encoding that identifies, for a given sequence, which nucleotide is aligned to a particular position. Thus, a feature vector can include a binary set of values, which may indicate - for each of the potential bases and for the gap - whether the aligned sequence includes the base (or gap) at the position. When condensed modified representations of the sequences are aligned and when a representation of a homopolymer is aligned to a given position, the feature vector may further identify a number of bases that were included in the homopolymer.

In some instances, a feature vector further includes one or more quality metrics. For example, a quality metric may be generated based on (for example) raw data (e.g., peak height and width) and empirical data that relates characteristics of raw data to error probabilities. In instances where a homopolymer was collapsed, a given nucleotide representation in an aligned set of sequence representations may correspond to multiple nucleotides from an original sequence, each of which may be associated with a separate quality metric. Thus, the feature vector may include one or more statistics generated based on the separate quality metrics (e.g., a mean, median, maximum or minimum) and potentially the feature vector includes the quality metric itself if a nucleotide in the aligned set of sequence representations represents only a single base (and not a homopolymer).

The feature vector may characterize each of a set of sequences, such that it may include (for example) multiple encodings of which base(s) are associated with a given position, multiple quality metrics, multiple base quantities, etc. For example, a feature vector may include a first set of values that corresponds to a first sequence (e.g., identifying a base and quality metric), a second set of values that corresponds to a second sequence, etc.

### II.E. Machine Learning Models

A machine learning model used to process feature vectors can include (for example) a recurrent neural network (e.g., including one or more long-short term memory (LSTM) cells or one or more gated recurrent units (GRUs)) or a convolutional neural network. For example, the model may include one or more layers each having one or more LSTM cells. Each LSTM cell can include a forget gate (that controls the degree to which values of a previous LSTM cell are to influence a current cell), one or more input gates (that control the extent to which input is to influence a current cell) and an output gate (that controls the extent to which one or more states of the cell are to be output). As another example, the model may include one or more layers each having one or more GRUs. Each GRU can include a forget game (that controls the degree to which information flows out of memory) and an update gate (that controls the degree to which states of a previous cell are stored in memory).

In some instances, a machine learning model can be a deep machine learning model which may include (for example) three or more, four or more, five or more, seven or more or ten or more layers; and/or the deep machine learning model may include three or more, four or more, five or more, seven or more or ten or more hidden layers (e.g., which may include one or more, two or more or three or more LSTM and/or GRU layers). For example, a recurrent network includes a recurrent (feedback) connection at a hidden layer, such that a deep recurrent network may include multiple (e.g., two or more, three or more, etc.) hidden layers that include one or more recurrent connections. A deep machine learning model may include (for example) two or more, three or more, four or more or five or more LSTM layers (each including one or more or two or more LSTM units) and/or two or more, three or more, four or more or five or more GRU layers (each including one or more or two or more GRUs)

The machine learning model (e.g., a recurrent neural network) may be configured to receive variable-length inputs, and/or preprocessing may be performed to conform a size of the feature vectors to a predefined and/or static input-data size (e.g., using padding). An input of the machine learning may have a dimension of [a, b], and an output of the machine learning model may have dimensions of [c, d], where (for example) a=c and/or b>d. An actual, maximum or potential input may correspond to a quantity of positions (e.g., representing bases and/or gaps) represented by a, and b may identify and/or may be based on a sum of a number of potential bases (e.g., four), a gap, a quantity of bases in a homopolymer and/or a quantity of one or more quality metrics.

The machine-learning model may be trained using supervised or unsupervised learning. For example, well-characterized cell lines may be used as samples with known ground-truth results to support supervised learning. In some instances, the model may be trained using data associated with one or more first subjects and subsequently used to process samples of one or more different second subject. As another example, unsupervised learning may be performed by using a clustering- or entropy-based optimization (e.g., that optimizes separation between clusters or input entropy).

The machine-learning model may be trained using a focal loss function due to a class imbalance across nucleotides, due to an imbalance in the prevalence of gaps relative to the prevalence of one or more nucleotides, and/or due to single-nucleotide instances being more common than homopolymers.

In some instances, a loss function may be defined to penalize any error in a binary sense. In some instances, a loss function may be defined with a term that scales based on a degree to which a predicted length differed from an absolute length. When a loss depends both on nucleotide and length predictions, the term(s) pertaining to nucleotide (or gap) predictions may be determined independently from the term(s) pertaining to length predictions. For example, if a model predicted that a given column represented a single adenine, and it instead included a single guanine, the loss may selectively penalize for the nucleotide error. In instances where a model predicts a nucleotide (or gap) and a length for each column, a loss function may be configured to weight the accuracy of the nucleotide prediction the same as the accuracy of the length prediction.

### III. Exemplary Sequence Assessment Network

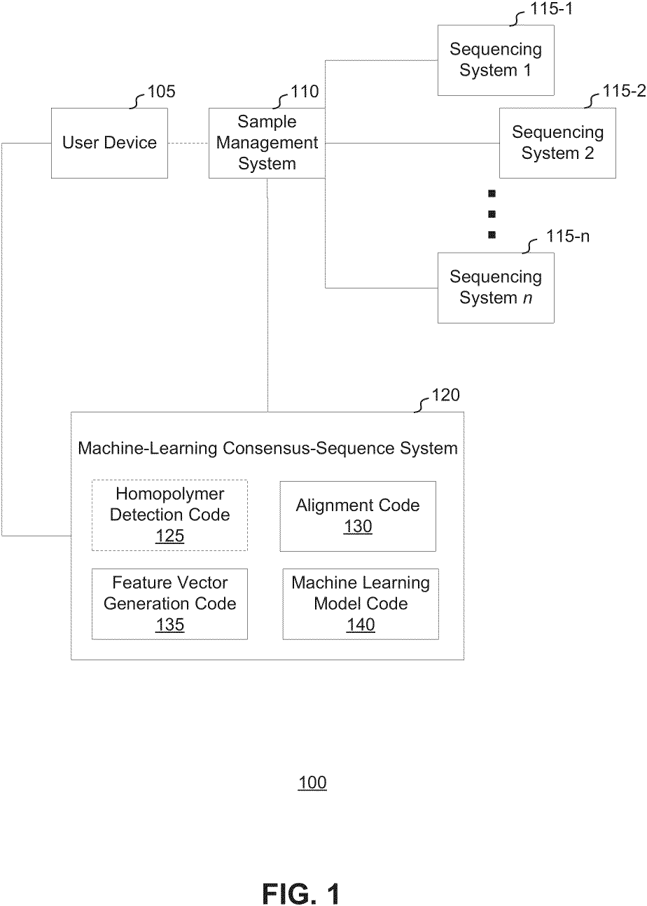

FIG. 1 depicts an exemplary sequence assessment network 100 for generating consensus sequences. A user device 105 may include a computing device associated with (e.g., owned by and/or used by) a user who may be or who may be affiliated with a medical care provider (e.g., a physician, nurse, doctor’s office, hospital, medical/clinical laboratory, etc.). User device 105 may receive input from a user that indicates that a sequencing assessment is to be performed for a particular subject. The input may identify (for example) a name of the particular subject, an alphanumeric identifier of the particular subject, residential address of the particular subject, demographic information of the particular subject (e.g., age, race, sex), one or more diagnoses of the subject, one or more potential diagnoses of the subject and/or one or more symptoms experienced by the subject. User device 105 may further receive input that indicates a type of sequencing analysis that is being requested (e.g., identifying a sequence of, any variants within and/or categorization of any variants within one or more genes, one or more chromosomes or genome). User device 105 may translate the input into (for example) entries into fields on one or more interfaces and/or content associated with one or more applications.

The field entries and/or content may be transmitted (e.g., via one or more web servers) to a sample management system 110 that may be configured to coordinate the collection of and analysis of a sample of the subject to identify the requested information. Sample management system 110 may be associated with a medical laboratory. Sample management system 110 may provide information to (or that may be conveyed to) one or more laboratory technicians that indicate that collection of a sample (e.g., a sample of a particular type) is requested and/or authorized and may further indicate a type of requested sample (e.g., blood or saliva), types of assessments that are requested (e.g., which may indicate a volume of a sample that is to be collected) and/or one or more volumes of samples that are requested.

The collected sample may be processed using one or more sequencing systems 115-1, 115-2, ... 115-n. Each of one or more sequencing systems 115-1, 115-2, ... 115-n may include one or more devices configured to process at least part of the sample to identify a sequence. The sequence may include a sequence corresponding to the request received by sample management system 110. In some instances, each sequencing system performs a different type of sequencing. A single sample or a single portion of a sample may be used multiple times to generate multiple predictions of the sequence and/or different portions of the sample may be used to generate different sequencing predictions. Each of one or more of sequencing systems 115-1, 115-2, ... 115-n may use a single-molecule sequencing technique, next-generation sequencing technique and/or a sequencing technique disclosed herein (e.g., in Section II.A). In some instances, each of multiple sequences are identified using two or more sequencing techniques, two or more single-molecule sequencing techniques, two or more next-generation sequencing techniques and/or two or more sequencing techniques disclosed herein.

Each sequence identified by each sequencing system 115-1, 115-2, ... 115-n may identify an ordered set of bases (e.g., nucleotides). Each of one, more or all of sequencing systems 115-1, 115-2, ... 115-n may use a sequencing technique and/or determine a sequence based on an approach as described in Section II.A. Each of one or more sequencing systems 115-1, 115-2, ... 115-n may further provide one or more quality metrics. For example, a sequencing system may use one or more look-up tables and/or algorithms to associate one or more raw-data characteristics with a confidence of a base prediction. For example, one or more intensities (e.g., amplitude), one or more widths, one or more skews and/or one or more peak quantities from an optical or electrical signal may correspond to a confidence of a nucleotide prediction. Thus, an output from a sequencing system 115-1, 115-2, ... 115-n may include an ordered set of nucleotides and a paired set of quality metrics (e.g., confidence scores).

The output from each sequencing system 115-1, 115-2, ... 115-n may be availed to (e.g., transmitted to) system management system 110. In some instances, each of one, more or all of sequencing systems 115-1, 115-2, ... 115-n are co-located (e.g., in a single building and/or at a single address) with sample management system 110. In some instances, each of one, more or all of sequencing systems 115-1, 115-2, ... 115-n are remote from sample management system 110 (e.g., such that a sample, or a part of a sample, may be shipped from a location associated with sample management system 110 to one or more other locations associated with one or more sequencing systems).

Sample management system 110 may, but need not, further process the outputs from the sequencing systems (e.g., to refine sequence data to one or more particular portions of a gene, one or more particular genes, one or more particular portions of the genome; to transform one or more quality metrics to a different scale or variable type; etc.). Sample management system 110 may transmit and/or otherwise avail the outputs and/or processed versions thereof to a machine-learning consensus-sequence system 120, which may include (for example) one or more remote computing systems, one or more remote servers, one or more cloud computing systems and/or one or more cloud servers. The transmission may occur via one or more web sites and/or one or more web portals. In some instances, the transmission is accompanied by additional information, such as information identifying and/or characterizing a subject and/or information identifying rationale for the sequencing analysis. In some embodiments, a sequencer may include both a sequencing system and a consensus-sequence system. For example, the sequencer may include one or more processors that have been configured to perform machine learning computations (e.g., NVIDIA GPUs, AMD GPUs, or dedicated machine learning CPUs).

Machine-learning consensus-sequence system 120 may collectively analyze the outputs from sequencing systems 115-1, 115-2, ... 115-n (or processed versions thereof) to identify a consensus sequence. The consensus sequence may include a single sequence generated based on a set of sequences. The set of sequences may include at least two, at least three or at least four sequences generated using different sequencing systems (e.g., sequencing systems 115-1, 115-2, ... 115-n) and/or using different sequencing techniques, and/or the set of sequences may include at least two, at least three or at least four sequences generated during different runs, at different times, using different portions of a sample and/or using different samples. The consensus sequence may be generated using one or more machine-learning models and/or via execution of one or more codes and/or one or more functions. Exemplary code and/or functions are depicted in FIG. 1, though it will be appreciated that other code and/or functions are not depicted. For example, machine-learning consensus-sequence system 120 may include code for an operating system.

Machine-learning consensus-sequence system 120 may, optionally, include homopolymer detection code 125, which may be configured to detect each homopolymer in each sequence (e.g., from sequencing systems 115-1, 115-2, ... 115-n). A sequence representation may include an ordered set of nucleotides and/or a modified version of an ordered set of nucleotides (e.g., as indicated in Section II.B). A modified version may collapse each homopolymer in an initial sequence to a single base. Homopolymer detection code 125 may identify any instance in which two or more (or three or more, four or more, etc.) consecutive nucleotides are the same nucleotide as a homopolymer. Homopolymer detection code 125 may further generate a modified sequence representation in which each detected homopolymer is replaced (e.g., by a single instance of the nucleotide of the homopolymer). In some instances, the modified sequence representation, another sequence representation or metadata may further indicate, for each homopolymer, a number of nucleotides that were included in the homopolymer and/or a quality metric for each nucleotide in the homopolymer. Homopolymer detection code 125 may identify, for each homopolymer, a quality statistic. For example, a quality statistic may include a mean, median, maximum or minimum quality metric across nucleotides in the homopolymer.

Machine-learning consensus-sequence system 120 may include an alignment code 130 that may align representations of the sequences and/or modified representations of the sequences relative to each other. An alignment may be performed in accordance with one or more alignment techniques and/or approaches as indicated in Section II.C. An alignment may be performed using a multiple-sequence-alignment technique, a progressive alignment technique, a consensus-alignment technique, a Hidden-Markov alignment technique, etc. The alignment may include introducing one or more gaps into the representations, shifting one or more parts of sequence representations, etc. Depending on a loss function or objective function, a gap may be preferentially added in favor of having inconsistency among aligned nucleotides (e.g., or at least a threshold degree of inconsistency).

A feature vector generation code 135 may be configured to use the aligned sequence representations to generate a set of features. Feature vectors may be generated in accordance with a technique and/or approach as described in Section II.A. In some instances, the aligned sequence representations correspond to a set of positions, and a feature vector is generated for each position. The feature vector may indicate (for example) an absolute or relative quantity of nucleotides (or representations thereof) aligned to the position are identified as a particular nucleotide. For example, the feature vector may indicate that 7 or 9 sequences included a cytosine at a particular location. In instances where an alignment aligns a collapsed and/or modified representation, a feature vector may indicate a quantity of bases represented for each position-sequence combination. A feature vector may include one or more quality metrics, which might correspond to a confidence of identifying one or more nucleotides.

A machine learning model code 140 can use the feature vectors (corresponding to multiple sequences) to predict a consensus sequence using a set of feature vectors (e.g., corresponding to multiple positions). The consensus sequence may be predicted using a machine-learning model (e.g., a model described in Section II.E), such as an LSTM neural network and/or deep neural network, or a combination of any of the neural networks described herein. The consensus sequence may include an ordered set of nucleotides.

Machine-learning consensus-sequence system 120 may transmit the consensus sequence to user device 105. In some instances, machine-learning consensus-sequence system 120, user device 105 and/or another system may compare the consensus sequence to one or more reference sequences to predict whether the consensus sequence includes any variants. A variant may include (for example) a single nucleotide polymorphism and/or a copy-number variation. When machine-learning consensus-sequence system 120 performs an analysis to detect any variants, a result of the variant analysis (e.g., that identifies any variant) can be transmitted to user device 105. A diagnosis, prognosis, treatment selection and/or other recommendation may then be informed using the result.

### IV. Exemplary Processing for Consensus-Sequence Identification

### IV.A. Using Representations of Individual Nucleotides

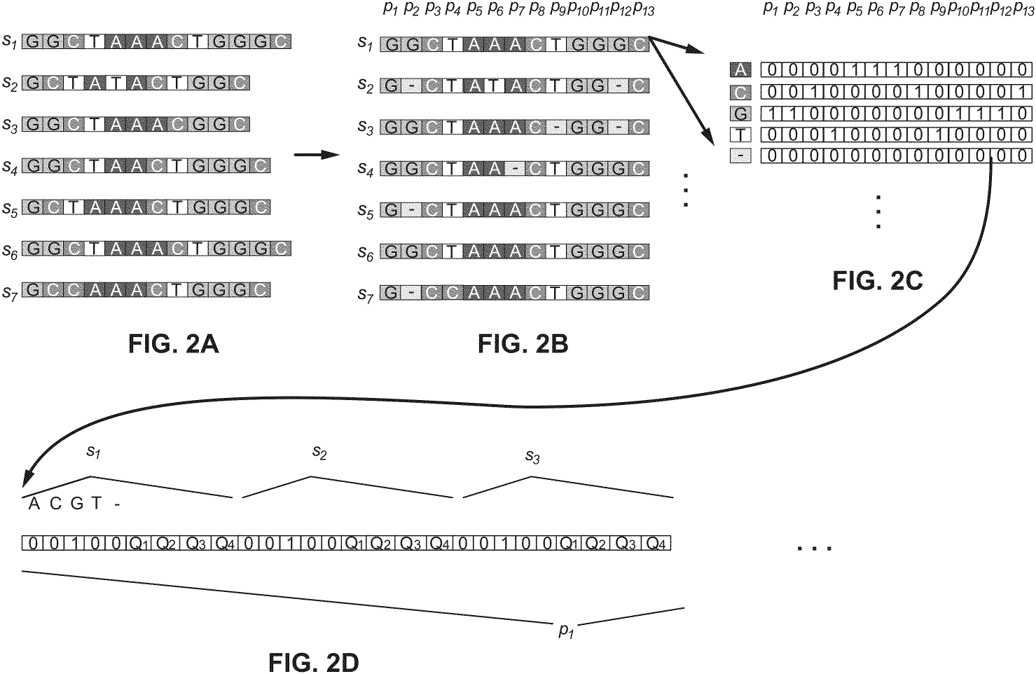

FIGS. 2A-2D illustrate exemplary processing of sequences to generate a consensus sequence. FIG. 2A depicts 7 exemplary sequences, s₁-s₇ of a particular (same) molecule. Each of s₁-s₇ is a sequence split. Split sequences can include a set of sequences corresponding to a same DNA fragment. The split sequences can be generated, for example, by sequencing the same molecule multiple times and/or by sequencing multiple copies of the same molecule. Each of the exemplary sequences s₁-s₇ may have been generated (for example) using one or more sequencing techniques, using one or more different sequencing machines and/or under the control of one or more different entities. Each of exemplary sequences s₁-s₇ includes an ordered set of nucleic acids. Each of the sequences may have been generated using one or more sequencing techniques, using one or more different sequencing machines, during different runs, at different times and/or under the control of a different entity. In some instances, each of one or more of the exemplary sequences s₁-s₇ may have been generated using a different portion of one or more amplified or cloned sequences. In some instances, each of one, more or all of the exemplary sequences s₁-s₇ may have been generated by processing a same portion of a sample and/or a same sample.

FIG. 2B shows an alignment result where the exemplary sequences s₁-s₇ are aligned to each other. The alignment result includes multiple gaps between sequential bases to account for potential deletions and/or insertions. The alignment may have been performed using one or more alignment techniques disclosed herein (e.g., in Section II.C). In the illustrated instance, generally, for each position, the position includes (across sequences) only a single nucleotide and potentially one or more gaps. However, with respect to one position (p₆), a particular sequence (s₂) includes a different base than is present in other sequences. It will be appreciated that configurations and/or hyperparameters of an alignment function may influence whether to accept nucleotide inconsistency across sequences (e.g., to a particular degree) rather than introducing one or more additional gaps to allow for the nucleotides to be separated to different positions.

FIG. 2C shows an exemplary representation of a particular aligned sequence. Specifically, for each position, a set of binary values are determined - each corresponding to a nucleotide base on a gap. The value can be set to 1 if the nucleotide at the position for the particular aligned sequence is equal to the corresponding base (or gap) and otherwise can be set to 0. Thus, in some instances, each position is associated with one, and only one, value of 1. These binary values may be determined for each sequence.

FIG. 2D shows part of a feature vector that includes binary values for each sequence at a particular position. In particular, the feature vector depicted in FIG. 2D corresponds to the first position, p₁. The depicted representations of the nucleotides correspond to the nucleotides at the first position p₁ in the first three sequences s₁-s₃, though it will be appreciated that the feature vector may represent nucleotides at the first position across all sequences s₁-s₇.

The depicted feature vector further includes, for each of the sequence reads, a set of quality metrics (Q₁-Q₄, which may be different values for different sequences). In some instances, the four scores in the depicted instance may correspond to a probability that a nucleotide in a corresponding position in a corresponding sequence is a particular nucleotide. For example, Q₃ may indicate a probability that a nucleotide is a guanine. Thus, a score may be highest in association for a nucleotide associated with a “1” binary value (e.g., such that, for sequence s₁ and at position p₁, the quality metric Q₃ may be higher than quality metrics Q₁, Q₂ and Q₄, given that it is predicted that the nucleotide for sequence s₁ and at position p₁ is a guanine).

In some instances, each quality metric reflects a degree to which a confidence in interpreting a portion of raw data that corresponds to a given type of nucleotide. For example, each of the four nucleotides may be associated with a particular wavelength and/or signal signature. If a signal clearly included a peak at a particular wavelength associated with cytosine and/or clearly included a signal signature of cytosine, a quality metric associated with cytosine may be high so as to indicate a high confidence in a prediction that the nucleotide was cytosine. Alternatively, if a signal clearly lacked a peak at the particular wavelength associated with cytosine and/or clearly lacked a signal signature of cytosine, a quality metric associated with cytosine may still be high so as to indicate a high confidence in a prediction that the nucleotide was not cytosine. Meanwhile, if a signal included a weak and/or broad peak at the particular wavelength associated with cytosine and/or included some weak representation of the signal signature of cytosine, a quality metric associated with cytosine may be low so as to indicate a low confidence in a prediction as to whether the nucleotide was cytosine. One or more quality metrics may be output by one or more devices that generate the corresponding sequence. A quality metric may be (for example) a binary, integer or real-number value.

The exemplary feature vector depicted (in part) in FIG. 2D corresponds to a single position. Each additional position can be associated with another corresponding feature vectors.

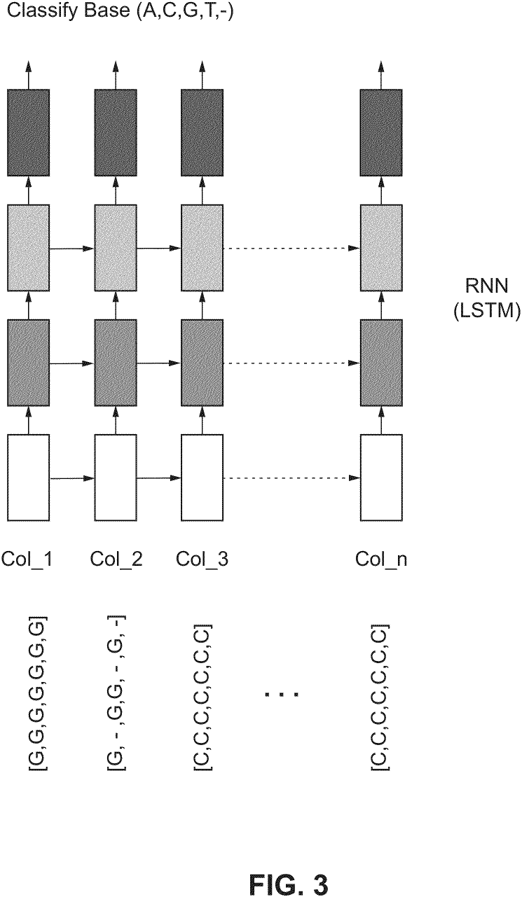

The feature vectors can then be input into a machine-learning model. FIG. 3 illustrates an exemplary neural network for processing a set of feature vectors to generate a result corresponding to a consensus sequence. For illustration simplicity, FIG. 3 shows the aligned bases as being input to the neural network instead of corresponding feature vectors, though it will be appreciated that in practice, the network may receive the feature vectors.

The depicted machine-learning model includes a deep recurrent neural network, and -in particular - a deep LSTM model. The model may be configured dynamically to receive input data having a size of [m, n], where m can be equal to a number of feature vectors (corresponding to a number of positions in an aligned sequence-representation data set) and n can be equal to a length of the feature vectors. The length of the feature vectors (n) may be defined to be equal to (or greater than or equal to) a product of: (1) a number of sequence data sets that are available (e.g., corresponding to a single particular molecule; and (2) a sum of a length of encoding of a nucleotide (e.g., which may be 4 so as to represent all potential bases or 5 so as to represent all potential bases plus a gap) plus a quantity of quality metrics pertaining to individual nucleotide identification. Thus, for the example illustrated in FIGS. 2A-2D, the length of each feature vector (n) may be 7*(4+4) = 56. It will be appreciated that a feature vector may be padded and/or subsampling may be used to achieve a target feature-vector length. For example, if a maximum number of split sequences was identified as being 100, a feature vector length can be defined as 100*(number of features per split sequence). In a situation where a number of split sequences is below 100, the vector may be zero padded to the feature vector length. In a situation where a number of actual split sequences is more than 100, subsampling may be performed to reduce the quantity to 100. In this manner, a size of a feature vector per column may be fixed.

Each node depicted in FIG. 3 may include an LSTM unit that receives data from a corresponding node in a lower layer (e.g., an input node or a node in a lower hidden layer) and from an adjacent (e.g., previous) node in a same layer. It will be appreciated that the LSTM network in FIG. 3 is exemplary and other neural networks are contemplated (e.g., a network that includes one or more Gated Recurrent Units, one or more bidirectional LSTM units, one or more bidirectional Gated Recurrent Units, etc.). The LSTM layers can thus pass select information representations across adjacent nodes, such that a prediction of a base at a given position may be informed based on a prediction of one or more bases at prior one or more prior positions. An output may include, for each position, a predicted consensus nucleotide (or gap). In some instances, an output further includes, for each position, a confidence metric corresponding to a confidence in the prediction.

The output may be post-processed to, for example, remove any gaps. The resulting sequence may then be identified as a consensus sequence.

The depicted instance corresponds to a network in which each of multiple LSTM layers are connected in a same direction. It will be appreciated that, in some instances, a bidirectional LSTM (or GRU) network may be alternatively used (e.g., where connections in alternating layers are in opposite directions).

### IV.B. Using Representations of Individual Nucleotides and Homopolymers

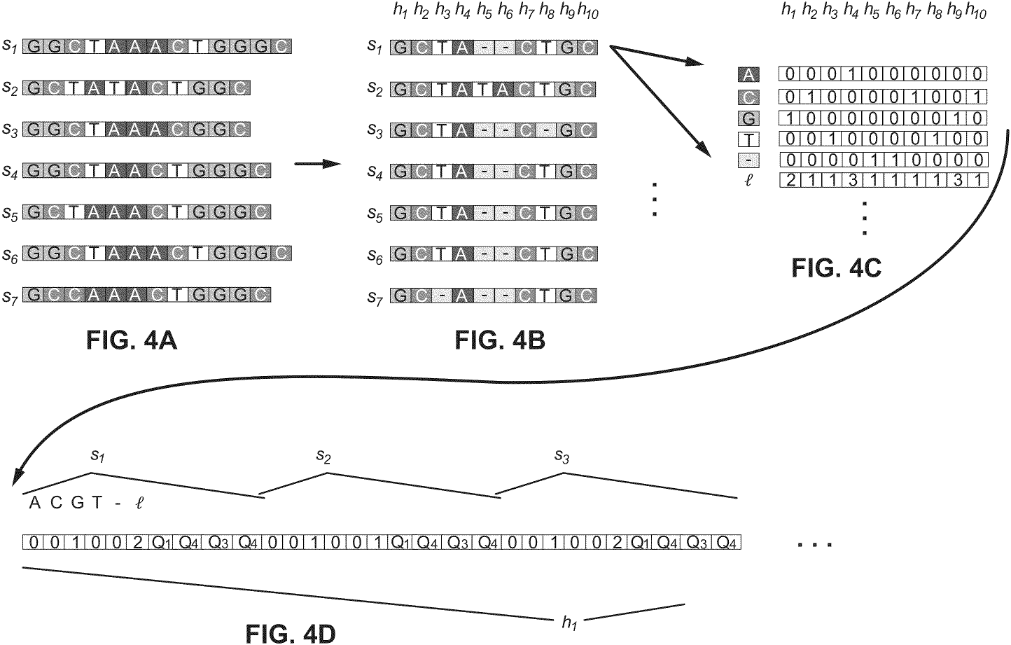

FIGS. 4A-4D illustrate another exemplary processing of sequences to generate a consensus sequence. The sequences identified in FIG. 4A are the same as the sequences identified in FIG. 2A. However, the representations of the sequences are generated differently, in that homopolymers are collapsed prior to aligning the sequences. Thus, each instance where successive nucleotides are identified as being the same nucleotide are modified to remove all but the first nucleotide. The sequence representations may then be aligned (e.g., as shown in FIG. 4B), which may be performed using one or more alignment techniques disclosed herein (e.g., in Section II.C). In the illustrated instance, for each position, the position includes (across sequences) only a single nucleotide and potentially one or more gaps.

FIG. 4C illustrates how each sequence may be represented via numeric values. In the illustrated case, at each position, five binary values are defined to indicate whether the nucleotide(s) at the position corresponds to any one of four nucleotides or a gap. A sixth “length” value indicates how many nucleotides are represented in the position. Thus, if a homopolymer that included 3 bases were collapsed to a single nucleotide representation, the length value may be set to 3 for the position.

A feature vector may be generated based on the representations of the set of collapsed sequences. (FIG. 4D.) As in the example of FIG. 2D, the feature vector may include one or more quality metrics. In contrast to the example of FIG. 2D, the feature vector also includes - for each sequence representation - the length value.

Notably, the quantity of feature vectors determined using the homopolymer-collapsed sequence representations (e.g., corresponding to FIGS. 4A-4D) may be smaller than the quantity of feature vectors determined using the uncollapsed sequence representations (e.g., corresponding to FIGS. 2A-2D). However, in the depicted instance, the length of the feature vectors determined using the homopolymer-collapsed sequence representations may be longer than the length of the feature vectors determined using the uncollapsed sequence representation (e.g., due to the added length values in the former case).

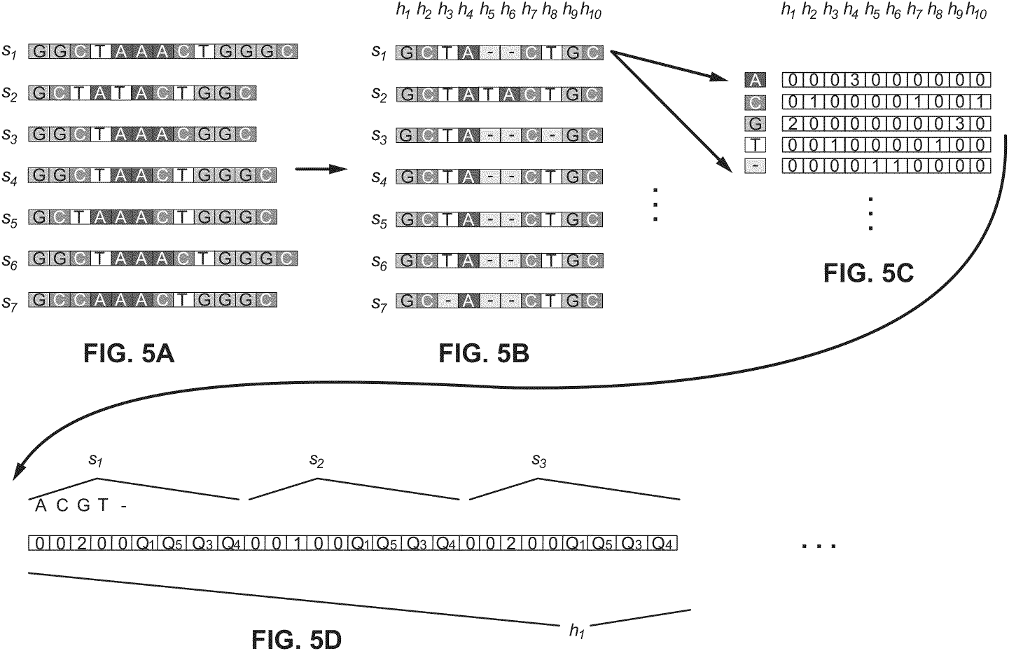

FIGS. 5A-5D yet another illustrate exemplary processing of sequences to generate a consensus sequence. The sequences identified in FIG. 5A are the same as the sequences identified in FIGS. 2A and 4A. The aligned sequence representations shown in FIG. 5B are the same as the sequences shown in FIG. 4B (such that homopolymers are collapsed). However, in the numeric sequence representations (shown in FIG. 5C), rather than using binary numbers to indicate which nucleotide (of gap) is present at the position, a value can indicate a number of nucleotides represented at the position. That is, rather than including a separate length value, the length value is used in lieu of a “1” at whichever nucleotide or gap position corresponds to the position. Thus, a representation of a homopolymer may have a nucleotide or gap value greater than 1. The feature vector (shown in FIG. 5D) then need not include a length value.

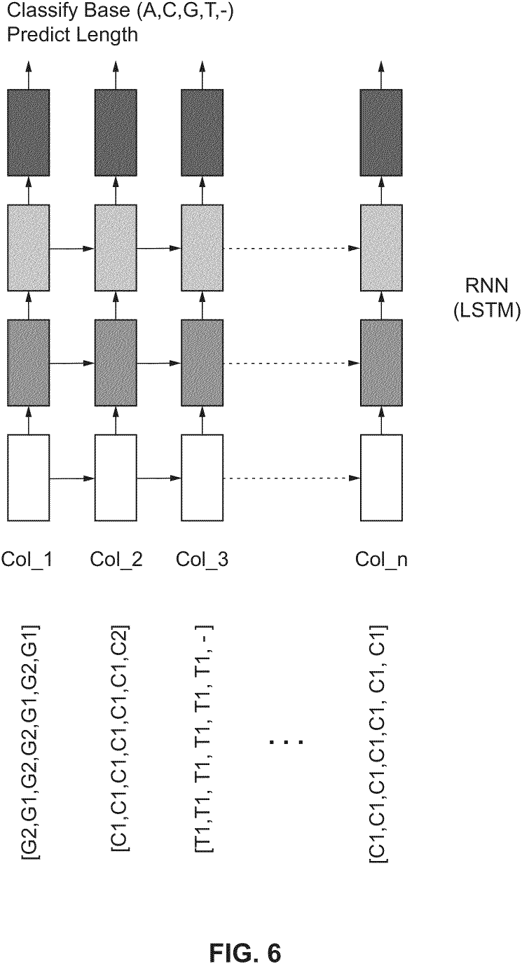

FIG. 6 illustrates an exemplary neural network for processing a representation of a set of sequences to generate a result corresponding to a consensus sequence. The depicted neural network may be configured to receive a set of feature vectors generated based on homopolymer-collapsed sequence representations (e.g., as illustrated in FIGS. 4A-4D or FIGS. 5A-5D). The neural network can be a same type of neural network and/or can include one or more same characteristics as described in relation to FIG. 3.

In this instance, an output of the model may include (at each position) a nucleotide identification (or gap) and also a length. For example, an output corresponding to column 1 may predict that the sequence begins with two guanines.

Post-processing may be used to delete any gaps in the output and to expand any instances in which it is predicted that a single position/column corresponds to multiple nucleotides. For example, post-processing may convert an output of “G2, C1, T1, -, C1” to “G, G, C, T, C”.

### V. Exemplary Sequence Assessment Process

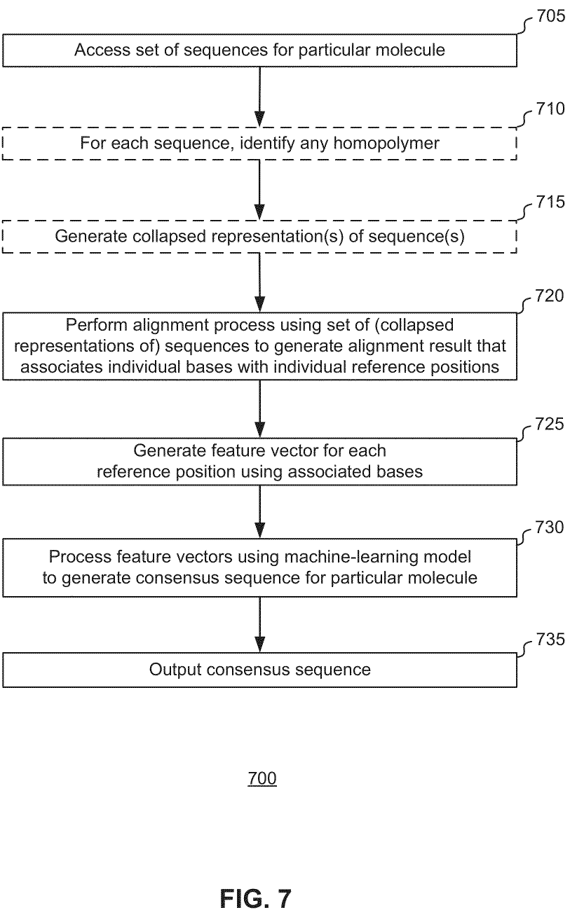

FIG. 7 illustrates a flowchart of an exemplary process 700 for processing a set of sequences to generate a consensus sequence. Process 700 begins at block 705, where a set of sequences are accessed. Each of the set of sequences may correspond to (e.g., exclusively correspond to) a particular molecule. Each of one, more or all of the set of sequences may have been generated by sequencing a same sample, via sequencing occurring at a different time, via sequencing occurring using different techniques and/or via sequencing occurring using different machine.

In some instances, process 700 includes blocks 710 and 715. In some instances, process 700 does not includes blocks 710 and 715. At block 710, each homopolymer in each of the set of sequences is identified. At block 715, for each sequence that includes a homopolymer, a collapsed representation of the sequence is generated. The collapsed representation can include an initial representation of the sequence (e.g., that includes identification of an ordered set of nucleotides) modified such that each homopolymer is represented by only a single nucleotide identifier. Thus, for example, an initial sequence that includes G, G, G may be modified to replace the three nucleotide identifiers with a single G. Metadata and/or another data structure may be used to track how many nucleotides are represented by a single value in the collapsed representation.

Thus, a sequence representation can include an ordered set of identifiers, which may include identifiers of nucleotides, identifiers of homopolymers, and/or identifiers of gaps. At block 720, an alignment process is performed using the set of sequences (or collapsed representations of the set of sequences) to generate an alignment result. The alignment result may identify, for each of a set of reference positions and for each sequence of the set of sequences, a base (or gap or homopolymer) that corresponds to the position. The alignment may be performed using an alignment technique disclosed herein (e.g., in Section II.C). The reference positions may include positions used for data processing (e.g., to facilitate alignment). Thus, for example, a reference position may be added or included when an alignment technique produces a prediction that there is sufficient inconsistency across identifiers within an intra-sequence portion of the sequences so as to potentially represent association with different nucleotides in a ground truth.

Each of the sequences may be represented using a vector or set of vectors (e.g., matrix). For example, each nucleotide or homopolymer may be represented using one-hot encoding (or other encoding) so as to include 4 or 5 binary numbers. In some instances, the vector(s) can further identify a quantity of nucleotides represented in a single position for a given sequence (e.g., a length).

At block 725, a feature vector may be generated for each reference position. The feature vector may be generated by (for example) appending a set of vectors (e.g., corresponding to one or more nucleotides, a gap, quality metrics and/or lengths) to generate a single vector. The feature vector may (e.g., alternatively) be generated by transforming two-dimensional matrices and/or multi-dimensional arrays (e.g., with one axis representing different nucleotides and potentially a gap) into a vector. Representations of each sequence may be concatenated and/or appended with each other, such that a single vector may represent data from each of the set of sequences with respect to a particular position. A different feature vector may be generated for each reference position.

At block 730, the feature vectors may be processed using a machine-learning model to generate a consensus sequence for a particular molecule. For example, each feature vector may be fed to a different input node of a neural network. The neural network may include (for example) a recurrent neural network, LSTM neural network and/or deep neural network. The neural network may include horizontal connections and forward connections. An output from the machine-learning model may predict, for each of the reference positions, a particular nucleotide, gap or homopolymer that corresponds to the position. With respect to a prediction that a given position corresponds to a particular homopolymer, the output may identify a length of the homopolymer and a nucleotide of the homopolymer.

In some instances, an output of the machine-learning model is post-processed. The post-processing may include expanding representations of homopolymers to identify each nucleotide and/or removing gaps.

At block 735, the consensus sequence is output. For example, the consensus sequence may be transmitted to another device and/or presented (e.g., via a display). The consensus sequence may further or additionally be stored (e.g., in association with an identifier a sample, subject, etc.).

A consensus sequence may be compared against one or more reference signals to detect any variant (e.g., single nucleotide polymorphism or copy-number variant) in the consensus sequence. The variant may facilitate diagnosis of a medical condition (e.g., using a look-up table and/or one or more rules) and/or identification of a treatment. In some instances, the consensus sequence can be associated with (e.g., in a look-up table) a medical condition associated with the sample that was sequenced, which may facilitate subsequent diagnoses of the medical condition.

### VI. Example

A unique barcode was attached to each DNA molecule from a sample before PCR amplification, such that variants from the sample could be distinguished from base changes occurring due to processing of the sample. Bases in sequence reads tagged with the barcode were then determined using sequencing (described in Section II.A.3).

A consensus sequence was then determined for each of one or more clusters of sequences. More specifically, a “cluster” of sequences was defined to include a particular number of sequences having a same tag and aligned in the same location, and a consensus sequence for the cluster was determined for each of the two techniques. This process was repeated for all clusters using different cluster sizes. Each technique was used to determine a consensus sequence for the cluster (and for the cluster size).

For each technique and for each cluster size, a consensus base-level Phred score was determined. The two techniques were: (1) a “Deep Consensus” technique performed actions in blocks 710-730 of process 700, as described herein; and (2) an “fgbio” technique that identifies consensus sequences for tagged molecules using fgbio’s CallMolecularConsensusReads. The consensus base-level Phred score was defined such that higher scores represent a lower probability of error and the converse. For example, a consensus base-level Phred score of 20 represents an accuracy of 99%.

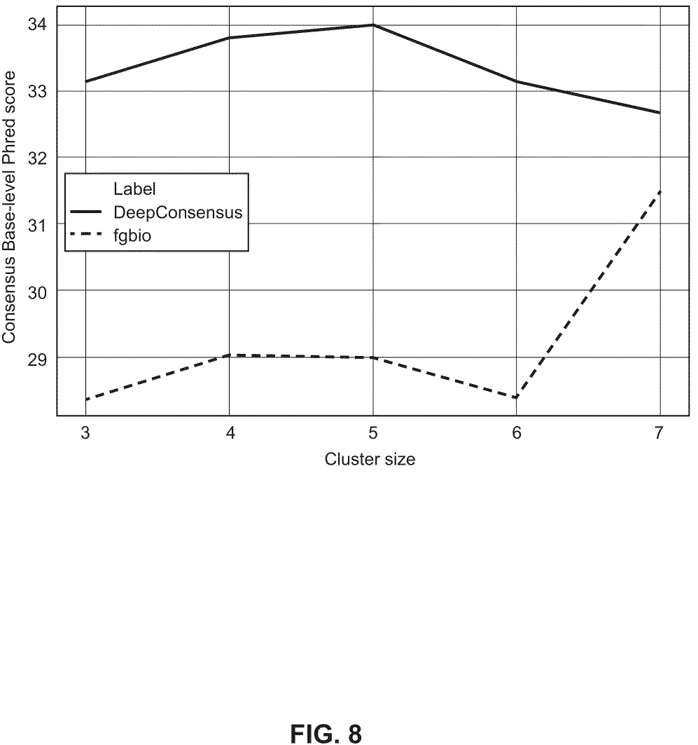

FIG. 8 shows the consensus base-level Phred scores across cluster sizes for each of the two techniques across different sizes of clusters. The scores generated by using the Deep Consensus technique were consistently better across the scores generated by using the fgbio technique. The improved performance of the Deep Consensus technique relative to the fgbio technique was particularly pronounced for small cluster sizes.

### VII. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed:

1. A method for generating a consensus sequence of a particular molecule, the method comprising:
accessing a set of sequences of the particular molecule, each of the set of sequences having been generated independently from other sequences in the set of sequences, each of the set of sequences including an ordered set of bases;
performing an alignment process using the set of sequences to generate an alignment result that associates, for each base of the ordered sets of bases of the sets of sequences, the base with a reference position from among a set of reference positions;
generating, for each reference position of the set of reference positions, a feature vector for the reference position that represents each base from the ordered sets of bases aligned to the reference position; and
processing the feature vectors for the set of references positions using a machine learning model to generate the consensus sequence for the particular molecule.

2. The method of claim 1, wherein performing the alignment processing includes performing multiple sequence alignment.

3. The method of claim 1, wherein, for each reference position of the set of reference positions, the feature vector includes, for each of the set of sequences, an indication as to which, if any, of the ordered set of bases is aligned to the reference position.

4. The method of claim 1, wherein, for each reference position of at least one reference position of the set of reference positions, the feature vector includes an indication that each of at least one of the set of sequences does not include a base aligned to the reference position.

5. The method of claim 1, further comprising, for each sequence of at least one of the set of sequences:
determining that the sequence includes one or more homopolymers, each of the one or more homopolymers including multiple sequential representations of a same base in the sequence; and
generating a collapsed representation of the sequence in which each of the one or more homopolymers is collapsed to a single base, wherein the alignment process is performed using the collapsed representations of the sequence.

6. The method of claim 5, wherein the collapsed representation includes, for each of the one or more homopolymers, an indication of a quantity of bases in the homopolymer.

7. The method of claim 1, wherein the machine learning model includes a recurrent neural network.

8. The method of claim 1, wherein the machine learning model includes one or more long short-term memory (LSTM) units.

9. The method of claim 1, further comprising:
accessing, for each sequence of at least some of the set of sequences, a quality metric for each of one or more bases of the ordered set of bases, wherein at least one of the generated feature vectors includes one or more quality values, each of the one or more quality values including or being based on the quality metric.

10. A system for generating a consensus sequence of a particular molecule, the system comprising:
one or more data processors; and
a non-transitory computer readable storage medium containing instructions which, when executed on the one or more data processors, cause the one or more data processors to perform a set of actions including:
accessing a set of sequences of the particular molecule, each of the set of sequences having been generated independently from other sequences in the set of sequences, each of the set of sequences including an ordered set of bases;
performing an alignment process using the set of sequences to generate an alignment result that associates, for each base of the ordered sets of bases of the sets of sequences, the base with a reference position from among a set of reference positions;
generating, for each reference position of the set of reference positions, a feature vector for the reference position that represents each base from the ordered sets of bases aligned to the reference position; and
processing the feature vectors for the set of references positions using a machine learning model to generate the consensus sequence for the particular molecule.

11. The system of claim 10, wherein performing the alignment processing includes performing multiple sequence alignment.

12. The system of claim 10, wherein, for each reference position of the set of reference positions, the feature vector includes, for each of the set of sequences, an indication as to which, if any, of the ordered set of bases is aligned to the reference position.

13. The system of claim 10, wherein, for each reference position of at least one reference position of the set of reference positions, the feature vector includes an indication that each of at least one of the set of sequences does not include a base aligned to the reference position.

14. The system of claim 10, wherein the set of actions further includes, for each sequence of at least one of the set of sequences:
determining that the sequence includes one or more homopolymers, each of the one or more homopolymers including multiple sequential representations of a same base in the sequence; and
generating a collapsed representation of the sequence in which each of the one or more homopolymers is collapsed to a single base, wherein the alignment process is performed using the collapsed representations of the sequence.

15. The system of claim 14, wherein the collapsed representation includes, for each of the one or more homopolymers, an indication of a quantity of bases in the homopolymer.

16. The system of claim 10, wherein the machine learning model includes a recurrent neural network.

17. The system of claim 10, wherein the machine learning model includes one or more long short-term memory (LSTM) units.

18. The system of claim 10, wherein the set of actions further includes:
accessing, for each sequence of at least some of the set of sequences, a quality metric for each of one or more bases of the ordered set of bases, wherein at least one of the generated feature vectors includes one or more quality values, each of the one or more quality values including or being based on the quality metric.

19. A computer-program product tangibly embodied in a non-transitory machine-readable storage medium, including instructions configured to cause one or more data processors to perform a set of actions including:
accessing a set of sequences of a particular molecule, each of the set of sequences having been generated independently from other sequences in the set of sequences, each of the set of sequences including an ordered set of bases;
performing an alignment process using the set of sequences to generate an alignment result that associates, for each base of the ordered sets of bases of the sets of sequences, the base with a reference position from among a set of reference positions;
generating, for each reference position of the set of reference positions, a feature vector for the reference position that represents each base from the ordered sets of bases aligned to the reference position; and
processing the feature vectors for the set of references positions using a machine learning model to generate a consensus sequence for the particular molecule.

20. The computer-program product of claim 19, wherein performing the alignment processing includes performing multiple sequence alignment.
